(19) United States
(12) Patent Application Publication
(10) Pub. No.: US 2011/0015241 A1
(43) Pub. Date: Jan. 20, 2011

(54) SUBSTITUTED HETEROCYCLES AND USE THEREOF

(75) Inventors: Michael Härter, Leverkusen (DE); Tobias Wunberg, Hinterbruhl (AT); Susanne Röhrig, Hilden (DE); Stefan Heitmeier, Wulfrath (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/301,978

(22) PCT Filed: May 25, 2007
(86) PCT No.: PCT/EP2007/004694

§ 371 Date: Sep. 30, 2010

(30) Foreign Application Priority Data
May 31, 2006 (DE) 10 2006 025 319.1

(51) Int. Cl. A61K 31/422; C07D 413/14; A61P 7/02

(52) U.S. Cl. 514/377; 548/233; 435/2

(57) ABSTRACT

The invention relates to substituted heterocycles, to processes for their preparation, to their use for the treatment and/or prophylaxis of diseases and to their use for preparing medicaments for the treatment and/or prophylaxis of diseases, in particular of thromboembolic disorders.

The invention relates to novel substituted heterocycles, to processes for their preparation, to their use for the treatment and/or prophylaxis of diseases and to their use for preparing medicaments for the treatment and/or prophylaxis of diseases, in particular of thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a joint reaction path, are distinguished. Here factor Xa, which is formed from the proenzyme factor X, plays a key role, since it connects the two coagulation paths. The activated serine protease Xa cleaves prothrombin to thrombin. The resulting thrombin, in turn, cleaves fibrinogen to fibrin. Subsequent crosslinking of the fibrin monomers causes formation of blood clots and thus haemostasis. In addition, thrombin is a potent effector of platelet aggregation which likewise contributes significantly to haemostasis.

Haemostasis is subject to a complex regulatory mechanism. Uncontrolled activation of the coagulant system or defective inhibition of the activation processes may cause formation of local thrombi or embolisms in vessels (arteries, veins, lymph vessels) or in heart cavities. This may lead to serious thromboembolic disorders. In addition, in the case of consumption coagulopathy, hypercoagulability may—systemically—result in disseminated intravascular coagulation. Thromboembolic complications furthermore occur in microangiopathic haemolytic anaemias, extracorporeal blood circulation, such as haemodialysis, and also in connection with prosthetic heart valves.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants, i.e. substances for inhibiting or preventing blood coagulation, which are known from the prior art, have various, often grave disadvantages. Accordingly, in practice, an efficient treatment method or prophylaxis of thromboembolic disorders is very difficult and unsatisfactory.

In the therapy and prophylaxis of thromboembolic disorders, use is firstly made of heparin, which is administered parenterally or subcutaneously. Owing to more favourable pharmacokinetic properties, preference is nowadays more and more given to low-molecular-weight heparin; however, even with low-molecular-weight heparin, it is not possible to avoid the known disadvantages described below, which are involved in heparin therapy. Thus, heparin is ineffective when administered orally and has a relatively short half-life. Since heparin inhibits a plurality of factors of the blood coagulation cascade at the same time, the action is non-selective. Moreover, there is a high risk of bleeding; in particular, brain haemorrhages and gastrointestinal bleeding may occur, which may result in thrombopenia, drug-induced alopecia or osteoporosis [Pschyrembel, Klinisches Wörterbuch, 257th edition, 1994, Walter de Gruyter Verlag, page 610, entry “Heparin”; Römpp Lexikon Chemie, Version 1.5, 1998, Georg Thieme Verlag Stuttgart, entry “Heparin”].

A second class of anticoagulants are the vitamin K antagonists. These include, for example, 1,3-indanediones, and especially compounds such as warfarin, phenprocoumon, dicumarol and other coumarin derivatives which inhibit the synthesis of various products of certain vitamin K-dependent coagulation factors in the liver in a non-selective manner. Owing to the mechanism of action, however, the onset of the action is very slow (latency to the onset of action 36 to 48 hours). It is possible to administer the compounds orally; however, owing to the high risk of bleeding and the narrow therapeutic index, a time-consuming individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683].

Recently, a novel therapeutic approach for the treatment and prophylaxis of thromboembolic disorders has been described. This novel therapeutic approach aims to inhibit factor Xa. Because of the central role which factor Xa plays in the blood coagulation cascade, factor Xa is one of the most important targets for anticoagulants [J. Hauptmann, J. Stürzebecher, Thrombosis Research 1999, 93, 203; S. A. V. Raghavan, M. Dikshit, “Recent advances in the status and targets of antithrombotic agents” Drugs Fut. 2002, 27, 669-683; H. A. Wieland, V. Laux, D. Kozian, M. Lorenz, “Approaches in anticoagulation: Rationales for target positioning” Curr. Opin. Investig. Drugs 2003, 4, 264-271; U. J. Ries, W. Wienen, “Serine proteases as targets for (online publication August 2004)antithrombotic therapy” Drugs Fut. 2003, 28, 355-370; L. -A. Linkins, J. I. Weitz, “New anticoagulant therapy” Annu. Rev. Med. 2005, 56, 63-77 (online publication August 2004)].

It has been shown that, in animal models, various both peptidic and nonpeptidic compounds are effective as factor Xa inhibitors. A large number of direct factor Xa inhibitors is already known [J. M. Walenga, W. P. Jeske, D. Hoppensteadt, J. Fareed, “Factor Xa Inhibitors: Today and beyond” Curr. Opin. Investig. Drugs 2003, 4, 272-281; J. Ruef, H. A. Katus, “New antithrombotic drugs on the horizon” Expert Opin. Investig. Drugs 2003, 12, 781-797; M. L. Quan, J. M. Smallheer, “The race to an orally active Factor Xa inhibitor: Recent advances” Curr. Opin. Drug Discovery & Development 2004, 7, 460-469; A. Casimiro-Garcia et al., “Progress in the discovery of Factor Xa inhibitors” Expert Opin. Ther. Patents 2006, 15, 119-145]. Nonpeptidic low-molecular-weight factor Xa inhibitors are also described, for example, in WO 06/002099 and WO 03/026652.

It is an object of the present invention to provide novel alternative compounds having a comparable or improved activity and improved solubility in aqueous solutions, for controlling disorders, in particular thromboembolic disorders, in humans and animals.

The invention provides compounds of the formula

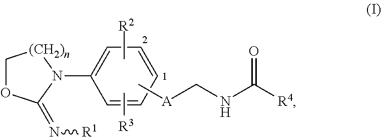

in which
n represents the number 1, 2 or 3,
A represents a 5-membered heteroaryl or a 5-membered heterocyclyl,
where heteroaryl and heterocyclyl are attached in the 1- or 2-position to the phenyl ring and heteroaryl and heterocyclyl for their part have a 1,3-attachment to the phenyl ring and the carbonylaminomethyl group,
and
where heteroaryl and heterocyclyl may be substituted by a substituent R⁸,
   where R⁸ is attached to the neighbouring atom of the atom to which the carbonylaminomethyl group is attached and has a 1,4-attachment to the phenyl ring
   and
   where the atom to which R⁸ is attached is a nitrogen or carbon atom
   and
   where R⁸ represents halogen, hydroxy, amino, C₁-C₄-alkyl, C₁-C₄-alkoxy, C₁-C₄-alkylamino, hydroxycarbonyl, aminocarbonyl, C₁-C₄-alkoxycarbonyl, C₁-C₄-alkylaminocarbonyl, aminosulphonyl, C₁-C₄-alkylaminosulphonyl or C₁-C₄-alkylsulphonyl,
      where alkyl, alkylamino and alkylaminosulphonyl may be substituted by a substituent, the substituent being selected from the group consisting of hydroxy, amino, C₁-C₄-alkoxy, C₁-C₄-alkylamino, hydroxycarbonyl, aminocarbonyl, C₁-C₄-alkoxycarbonyl, C₁-C₄-alkylaminocarbonyl and a 5- or 6-membered heterocyclyl attached via a nitrogen atom,
      and
      where alkylaminocarbonyl may be substituted by a substituent, the substituent being selected from the group consisting of hydroxy, amino, C₁-C₄-alkylamino and a 5- or 6-membered heterocyclyl attached via a nitrogen atom,
R¹ represents hydrogen, cyano, hydroxy, C₁-C₄-alkyl, C₁-C₄-alkylcarbonyl, C₃-C₇-cycloalkylcarbonyl, phenylcarbonyl, 4- to 7-membered heterocyclylcarbonyl or 5- or 6-membered heteroarylcarbonyl,
R² represents hydrogen, fluorine, chlorine, cyano, hydroxy, amino, trifluoromethyl, trifluoromethoxy, C₁-C₄-alkyl, C₁-C₄-alkoxy, C₁-C₄-alkoxymethyl, C₁-C₄-alkylamino, C₃-C₆-cycloalkyl, aminocarbonyl, C₁-C₄-alkoxycarbonyl or C₁-C₄-alkylaminocarbonyl,
R³ represents hydrogen, fluorine, chlorine, cyano, hydroxy, amino, trifluoromethyl, trifluoromethoxy, C₁-C₄-alkyl, C₁-C₄-alkoxy, C₁-C₄-alkoxymethyl, C₁-C₄-alkylamino, C₃-C₆-cycloalkyl, aminocarbonyl, C₁-C₄-alkoxycarbonyl or C₁-C₄-alkylaminocarbonyl,
R⁴ represents a group of the formula

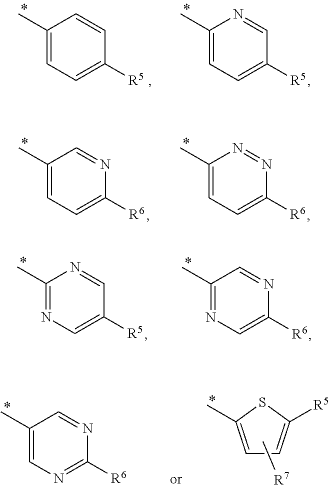

where
* is the point of attachment to the carbonyl group,
R⁵ represents hydrogen, fluorine, chlorine, cyano, ethynyl, C₁-C₄-alkyl, C₁-C₄-alkoxy or C₃-C₆-cycloalkyl,
R⁶ represents hydrogen, amino, C₁-C₄-alkyl, C₁-C₄-alkylamino or C₃-C₆-cycloalkyl,
and
R⁷ represents hydrogen, fluorine, chlorine, amino or C₁-C₄-alkyl,
   and their salts, their solvates and the solvates of their salts.

Compounds according to the invention are the compounds of the formula (I) and their salts, solvates and solvates of the salts, the compounds, comprised by formula (I), of the formulae mentioned below and their salts, solvates and solvates of the salts and the compounds, comprised by formula (I), mentioned below as embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified differently, the substituents have the following meanings:

Alkyl per se and “alk” and “alkyl” in alkoxy, alkylamino, alkoxycarbonyl, alkylaminocarbonyl, alkylaminosulphonyl and alkylsulphonyl represents a straight-chain or branched alkyl radical having generally 1 to 4, preferably 1 or 2, carbon atoms, by way of example and by way of preference methyl, ethyl, n-propyl, isopropyl and tert-butyl.

By way of example and by way of preference, alkoxy represents methoxy, ethoxy, n-propoxy, isopropoxy and tert-butoxy.

Alkylamino represents an alkylamino radical having one or two alkyl substituents (selected independently of one another), by way of example and by preference methylamino, ethylamino, n-propylamino, isopropylamino, tert-butylamino, N,N-dimethylamino, N,N-diethylamino, N-ethyl-N-methylamino, N-methyl-N-n-propylamino, N-isopropyl-N-n-propylamino and N-tert-butyl-N-methyl-amino. By way of example, C₁-C₃-alkylamino represents a monoalkylamino radical having 1 to 3 carbon atoms or represents a dialkylamino radical having in each case 1 to 3 carbon atoms per alkyl substituent.

By way of example and by way of preference alkoxycarbonyl represents methoxycarbonyl, ethoxycarbonyl, n-propoxycarbonyl, isopropoxycarbonyl and tert-butoxycarbonyl.

Alkylaminocarbonyl represents an alkylaminocarbonyl radical having one or two alkyl substituents (selected independently of one another), by way of example and by way of preference methylaminocarbonyl, ethylaminocarbonyl, n-propylaminocarbonyl, isopropylaminocarbonyl, tert-butylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N-tert-butyl-N-methylaminocarbonyl. By way of example, C₁-C₃-alkylaminocarbonyl represents a monoalkylaminocarbonyl radical having 1 to 3 carbon atoms or represents a dialkylaminocarbonyl radical having in each case 1 to 3 carbon atoms per alkyl substituent.

Alkylaminosulphonyl represents an alkylaminosulphonyl radical having one or two alkyl substituents (selected independently of one another), by way of example and by way of preference methylaminosulphonyl, ethylaminosulphonyl, n-propylaminosulphonyl, isopropylaminosulphonyl, tert-butylaminosulphonyl, N,N-dimethylaminosulphonyl, N,N-diethylaminosulphonyl, N-ethyl-N-methylaminosulphonyl, N-methyl-N-n-propylaminosulphonyl, N-isopropyl-N-n-propylamino-sulphonyl and N-tert-butyl-N-methylaminosulphonyl. By way of example, C₁-C₃-alkylamino-sulphonyl represents a monoalkylaminosulphonyl radical having 1 to 3 carbon atoms or represents a dialkylaminosulphonyl radical having in each case 1 to 3 carbon atoms per alkyl substituent.

By way of example and by way of preference alkylsulphonyl represents methylsulphonyl, ethylsulphonyl, n-propylsulphonyl, isopropylsulphonyl and tert-butylsulphonyl

Cycloalkyl represents a cycloalkyl group having generally 3 to 7 carbon atoms, preferably 3 to 5 carbon atoms, by way of example and by way of preference cyclopropyl, cyclobutyl, cyclopentyl, cyclohexyl and cycloheptyl.

Heterocyclyl represents a monocyclic radical having 5 or 6 ring atoms and up to 3, preferably up to 2, heteroatoms and/or heterogroups from the group consisting of N, O, S, SO, SO₂. The heterocyclyl radicals can be saturated or partially unsaturated. Preference is given to heterocyclyl radicals having up to two heteroatoms from the group consisting of O, N and S, such as, by way of example and by way of preference, tetrahydrofuranyl, pyrrolidinyl, pyrrolinyl, isoxazolinyl and morpholinyl.

Heteroaryl represents an aromatic monocyclic radical having 5 ring atoms and up to 4 heteroatoms from the group consisting of S, O and N, by way of example and by way of preference thienyl, furyl, pyrrolyl, thiazolyl, oxazolyl, isoxazolyl, isothiazolyl, imidazolyl and pyrazolyl.

If radicals in the compounds according to the invention are substituted, the radicals can, unless specified otherwise, be mono- or polysubstituted. In the context of the present invention, the meanings of all radicals which occur more than once are independent of one another. Substitution with one, two or three identical or different substituents is preferred. Very particular preference is given to substitution with one substituent.

In the formulae of the group which may represent R⁴, the end point of the line next to a * does not represent a carbon atom or a CH₂ group, but is part of the bond to the atom to which R⁴ is attached.

In the formulae of the group which may represent A, the end point of the line next to a #1 or #2 does not represent a carbon atom or a CH₂ group, but is part of the bond to the atom to which A is attached.

Preference is given to compounds of the formula (I) in which
n is the number 1, 2 or 3,
A represents a 5-membered heteroaryl or partially unsaturated 5-membered heterocyclyl,
where heteroaryl and heterocyclyl are attached in the 1- or 2-position to the phenyl ring and heteroaryl and heterocyclyl for their part have a 1,3-attachment to the phenyl ring and the carbonylaminomethyl group,
and
where heteroaryl and heterocyclyl may be substituted by a substituent R⁸,
   where R⁸ is attached to the neighbouring atom of the atom to which the carbonylaminomethyl group is attached and has a 1,4-attachment to the phenyl ring
   and
   where the atom to which R⁸ is attached is a nitrogen or carbon atom
   and
   where R⁸ represents amino, C₁-C₄-alkyl, C₁-C₄-alkoxy, C₁-C₄-alkoxymethyl, C₁-C₄-alkylamino, C₁-C₄-alkylaminomethyl, hydroxycarbonyl, hydroxycarbonylmethyl, hydroxycarbonylethyl, aminocarbonyl, aminocarbonylmethyl, aminocarbonylethyl, C₁-C₄-alkoxycarbonyl, C₁-C₄-alkoxycarbonylmethyl, C₁-C₄-alkoxycarbonylethyl, C₁-C₄-alkylaminocarbonyl, C₁-C₄-alkylaminocarbonylmethyl, C₁-C₄-alkylaminocarbonylethyl, aminosulphonyl, C₁-C₄-alkylaminosulphonyl or C₁-C₄-alkylsulphonyl,
      where alkyl may be substituted by a substituent, the substituent being selected from the group consisting of hydroxy and amino,
      and
      where ethylaminocarbonyl and propylaminocarbonyl may be substituted by a substituent, the substituent being selected from the group consisting of hydroxy, amino and C₁-C₄-alkylamino,
R¹ represents hydrogen, cyano, hydroxy or C₁-C₄-alkyl,
R² represents hydrogen, fluorine, chlorine, cyano, C₁-C₄-alkyl or C₁-C₄-alkoxy,
R³ represents hydrogen, fluorine, chlorine, cyano, hydroxy, C₁-C₄-alkyl, C₁-C₄-alkoxy, C₁-C₄-alkoxymethyl, cyclopropyl, aminocarbonyl, C₁-C₄-alkoxycarbonyl or C₁-C₄-alkylaminocarbonyl,
R⁴ represents a group of the formula

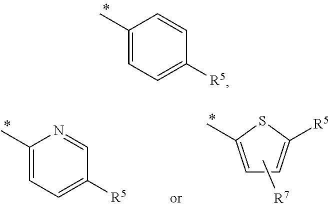

where
* is the point of attachment to the carbonyl group,
R⁵ represents fluorine, chlorine, ethynyl, methyl or methoxy,
and
R⁷ represents hydrogen,
   and their salts, their solvates and the solvates of their salts.

Preference is also given to compounds of the formula (I) in which
n represents the number 1 or 2,
A represents a group of the formula

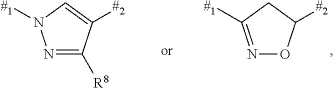

where
#1 is the point of attachment to the phenyl ring and is attached in the 1-position to the phenyl ring,
#2 is the point of attachment to the carbonylaminomethyl group,
R⁸ represents hydrogen, C₁-C₄-alkyl, C₁-C₄-alkoxy, C₁-C₄-alkoxymethyl, C₁-C₄-alkylamino, C₁-C₄-alkylaminomethyl, hydroxycarbonyl, hydroxycarbonylmethyl, aminocarbonyl, aminocarbonylmethyl, C₁-C₄-alkoxycarbonyl, C₁-C₄-alkoxycarbonylmethyl, C₁-C₄-alkylaminocarbonyl or C₁-C₄-alkylaminocarbonylmethyl,
   where alkyl may be substituted by a substituent, where the substituent is selected from the group consisting of hydroxy and amino,
   and
   where ethylaminocarbonyl may be substituted by a substituent, where the substituent is selected from the group consisting of hydroxy, amino and C₁-C₄-alkylamino,
R¹ represents hydrogen,
R² represents hydrogen or fluorine,
R³ represents hydrogen, fluorine, chlorine, cyano, methyl, ethyl, n-propyl, isopropyl, methoxy, ethoxy, methoxymethyl or cyclopropyl,
R⁴ represents a group of the formula

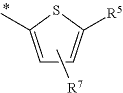

where
* is the point of attachment to the carbonyl group,
R⁵ represents fluorine, chlorine or methyl,
and
R⁷ represents hydrogen,
   and their salts, their solvates and the solvates of their salts.

Preference is also given to compounds of the formula (I) in which
n represents the number 1
A represents a group of the formula

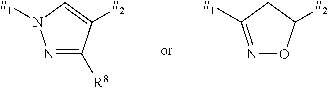

where
#1 is the point of attachment to the phenyl ring and is attached in the 1-position to the phenyl ring,
#2 is the point of attachment to the carbonylaminomethyl group,
R⁸ represents hydrogen, hydroxymethyl, aminomethyl, C₁-C₄-alkyl, C₁-C₄-alkoxy, C₁-C₄-alkoxymethyl, C₁-C₄-alkylaminomethyl, hydroxycarbonyl, aminocarbonyl, C₁-C₄-alkoxycarbonyl, C₁-C₄-alkylaminocarbonyl, hydroxyethylaminocarbonyl or C₁-C₄-alkylaminoethylaminocarbonyl,
R¹ represents hydrogen,
R² represents hydrogen or fluorine,
R³ represents hydrogen, fluorine, chlorine, methyl or methoxy,
R⁴ represents a group of the formula

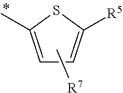

where
* is the point of attachment to the carbonyl group,
R⁵ represents chlorine,
and
R⁷ represents hydrogen,
   and their salts, their solvates and the solvates of their salts.

Preference is also given to compounds of the formula (I) in which n represents the number 1.

Preference is also given to compounds of the formula (I) in which A represents a group of the formula

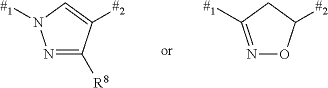

where
#1 is the point of attachment to the phenyl ring and is attached in the 1-position to the phenyl ring,
#2 is the point of attachment to the carbonylaminomethyl group,
and
R⁸ represents hydrogen, hydroxymethyl, aminomethyl, hydroxycarbonyl, aminocarbonyl, C₁-C₄-alkoxycarbonyl or C₁-C₄-alkylaminocarbonyl.

Preference is also given to compounds of the formula (I) in which R¹ represents hydrogen.

Preference is also given to compounds of the formula (I) in which R² represents hydrogen.

Preference is also given to compounds of the formula (I) in which R³ represents hydrogen, fluorine, chlorine, methyl or methoxy.

Preference is also given to compounds of the formula (I) in which R³ represents hydrogen.

Preference is also given to compounds of the formula (I) in which R² and R³ represent hydrogen.

Preference is also given to compounds of the formula (I) in which R² represents hydrogen and R³ represents fluorine.

Preference is also given to compounds of the formula (I) in which R⁴ represents a group of the formula

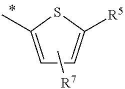

where * is the point of attachment to the carbonyl group, R⁵ represents chlorine and R⁷ represents hydrogen.

The individual radical definitions given in the respective combinations or preferred combinations of radicals are, independently of the particular given combinations of radicals, also replaced by any radical definitions of other combinations.

Very particular preference is given to combinations of two or more of the preferred ranges mentioned above.

The invention furthermore provides a process for preparing the compounds of the formula (I), or their salts, their solvates or the solvates of their salts, wherein

[A] the compounds of the formula

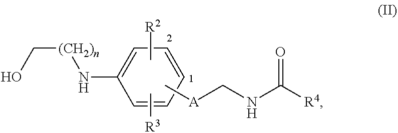

in which n, A, R², R³ and R⁴ have the meaning given above,
are reacted in an inert solvent in the presence of an acid with cyanogen bromide to give compounds of the formula (I), in which R¹ represents hydrogen,
or
[B] the compounds of the formula

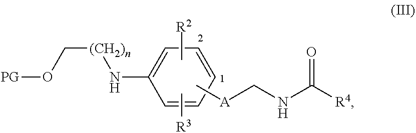

in which n, A, R², R³ and R⁴ have the meaning given above and
PG represents a hydroxy protective group, preferably trimethylsilyl or tert-butyldimethylsilyl,
are, in a three-step process, initially reacted in an inert solvent with cyanogen bromide, preferably in the presence of a base, to give compounds of the formula

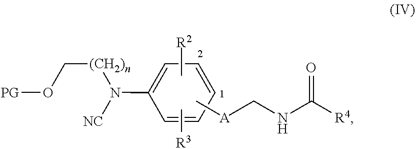

in which n, A, R², R³ and R⁴ have the meaning given above, and
PG represents a hydroxy protective group, preferably trimethylsilyl or tert-butyldimethylsilyl,
and then, by removal of the protective group PG, converted into compounds of the following formula

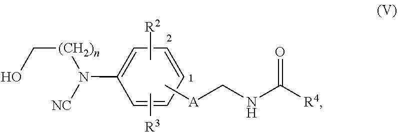

in which n, A, R², R³ and R⁴ have the meaning given above,
and, in the third step, the compounds of the formula (V) are cyclized in an inert solvent in the presence of an acid to give compounds of the formula (I), in which R¹ represents hydrogen, where the removal of the protective group and the cyclization are preferably carried out in one reaction step,
or
[C] the compounds of the formula (II) are reacted in the first step with compounds of the formula

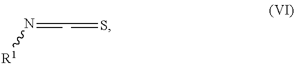

in which
R¹ represents C₁-C₄-alkyl, C₁-C₄-alkylcarbonyl, C₃-C₇-cycloalkylcarbonyl, phenylcarbonyl, 4- to 7-membered heterocyclylcarbonyl or 5- or 6-membered heteroarylcarbonyl,
and cyclized in the second step,
or
[D] the compounds of the formula (II) are reacted with compounds of the formula

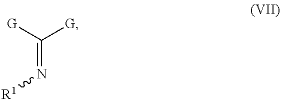

in which
R¹ represents cyano or C₁-C₄-alkyl and
G represents a leaving group, preferably phenoxy or methylthio,
or
[E] the compounds of the formula (I), in which R¹ represents hydrogen are reacted with hydroxylamine hydrochloride to give compounds of the formula (I), in which R¹ represents hydroxy.

The compounds of the formula (I), in which R¹ represents hydrogen can, if appropriate, be converted with the appropriate solvents and/or bases or acids into their salts, their solvates and/or the solvates of their salts.

The free base of the salts can be obtained, for example, by chromatography on a reversed-phase column using an acetonitrile/water gradient with an added base, in particular by using an RP18 Phenomenex Luna C18(2) column and diethylamine base, or by dissolving the salts in an organic solvent and extracting with aqueous solutions of basic salts such as sodium bicarbonate.

In an alternative process, the salts are dissolved in water and the base is precipitated by addition of sodium bicarbonate solution.

The invention furthermore provides a process for preparing the compounds of the formula (I) of their solvates wherein salts of the compounds or solvates of the salts of the compounds are converted into the compounds by chromatography with an added base.

The reaction according to process [A] is generally carried out in inert solvents, preferably in a temperature range of from −20° C. to 50° C. at atmospheric pressure.

Inert solvents are, for example, tetrahydrofuran, dichloromethane or acetonitrile or mixtures of these solvents.

Acids are, for example, strong inorganic or organic acids, such as hydrogen fluoride, hydrogen chloride, hydrogen bromide, methanesulphonic acid, trifluoromethanesulphonic acid or trifluoroacetic acid.

The reaction of the first step according to process [B] is generally carried out in inert solvents, preferably in a temperature range of from −20° C. to 50° C. at atmospheric pressure.

Inert solvents are, for example, tetrahydrofuran, dichloromethane or acetonitrile or mixtures of these solvents.

Bases are, for example, inorganic bases, such as alkali metal or alkaline earth metal carbonates or bicarbonates, such as lithium carbonate, sodium carbonate, potassium carbonate, calcium carbonate or caesium carbonate or sodium bicarbonate or potassium bicarbonate, or alkali metal hydrides, such as sodium hydride.

The removal of trimethylsilyl or tert-butyldimethylsilyl as preferred hydroxy protective groups (PG) in the second step according to process [B] is generally carried out in tetrahydrofuran as solvent, preferably with the aid of tetra-n-butylammonium fluoride (TBAF), preferably in a temperature range of from 0° C. to 40° C. at atmospheric pressure.

The reaction of the third step according to process [B] is generally carried out in inert solvents, preferably in a temperature range of from −20° C. to 50° C. at atmospheric pressure.

Inert solvents are, for example, tetrahydrofuran, dichloromethane or acetonitrile or mixtures of these solvents.

Acids are, for example, strong inorganic or organic acids, such as hydrogen fluoride, hydrogen chloride, hydrogen bromide, methanesulphonic acid, trifluoromethanesulphonic acid or trifluoroacetic acid.

The reaction of the second and third step according to process [B] is particularly preferably carried out using an acid-labile hydroxy protection group, such as, for example trimethylsilyl or tert-butyl-dimethylsilyl, in the presence of an excess of acid as a one-pot reaction, in inert solvents, preferably in a temperature range of from −20° C. to 50° C. at atmospheric pressure, without isolation of the intermediate of the compounds of the formula (V).

Inert solvents are, for example, tetrahydrofuran, dichloromethane or acetonitrile or mixtures of these solvents.

Acids are, for example, strong inorganic or organic acids, such as hydrogen fluoride, hydrogen chloride, hydrogen bromide, methanesulphonic acid, trifluoromethanesulphonic acid or trifluoroacetic acid.

The reaction of the first step according to process [C] is generally carried out analogously to processes known from the literature, as described, for example, in Hetenyi, et al., J. Org. Chem. 2003, 68, 2175-2182, D. Douglass, J. Amer. Chem. Soc. 1934, 56, 719, F. B. Dains et al., J. Amer. Chem. Soc. 1925, 47, 1981-1989 or F. B. Dains et al., J. Amer. Chem. Soc. 1922, 44, 2637-2643.

The reaction of the second step according to process [C] is generally carried out analogously to processes known from the literature, as described, for example, in T. Shibanuma, M. Shiono, T. Mukaiyama, Chem. Lett. 1977, 575-576.

The reaction according to process [D] is generally carried out analogously to processes known from the literature, as described, for example, in N. Maezaki, A. Furusawa, S. Uchida, T. Tanaka, Tetrahedron 2001, 57, 9309-9316, G. Berecz, J. Reiter, G. Argay, A. Kalman, J. Heterocycl. Chem. 2002, 39, 319-326, R. Evers, M. Michalik, J. Prakt. Chem. 1991, 333, 699-710, R. Mohr, A. Buschauer, W. Schunack, Arch. Pharm. (Weinheim Ger) 1988, 321, 221-227, P. J. Garratt, et al., Tetrahedron 1989, 45, 829-834 or V. A. Vaillancourt et al., J. Med. Chem. 2001, 44, 1231-1248.

The reaction according to process [E] is generally carried out analogously to processes known from the literature, as described, for example, in G. Zinner, G. Nebel, Arch. Pharm. Ber. Dtsch. Ges. 1970, 303, 385-390.

The compounds of the formulae (VI) and (VII) are known or can be synthesized by known processes from the appropriate starting materials.

The compounds of the formula (III) are known or can be prepared from the compounds of the formula (II) by introducing the protective group PG under conditions known to the person skilled in the art.

The introduction of trimethylsilyl or tert-butyldimethylsilyl as preferred hydroxy protective groups (PG) is generally carried out by reaction with trimethylsilyl chloride, tert-butyldimethylsilyl chloride or tert-butyldimethylsilyl trifluoromethanesulphonate in tetrahydrofuran, dimethylformamide or dichloromethane as solvent, preferably in the presence of imidazole or 2,6-dimethylpyridine, preferably in a temperature range of from 0° C. to 40° C. at atmospheric pressure.

The compounds of the formula (II) are known or can be prepared by reacting compounds of the formula

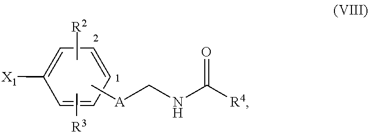

in which
A, R², R³ and R⁴ have the meaning given above and
X¹ represents bromine or iodine
with compounds of the formula

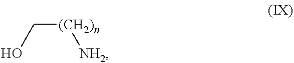

in which n has the meaning given above.

The reaction is generally carried out in inert solvents with addition of a copper(I) salt, a base and a diamine ligand, preferably in a temperature range of from 60° C. to reflux of the solvent at atmospheric pressure.

Inert solvents are, for example, aproptic solvents, such as toluene, dioxane, tetrahydrofuran or dimethylformamide; preference is given to dioxane.

Copper(I) salts are, for example, copper(I) iodide, copper(I) chloride or copper(I) oxide; preference is given to copper(I) iodide.

Bases are, for example, potassium phosphate, potassium carbonate or caesium carbonate; preference is given to potassium phosphate.

Diamine ligands are, for example, 1,2-diamines, such as N,N′-dimethylethylenediamine.

The compounds of the formula (VIII) are known or can be synthesized from the appropriate starting materials by processes, known to the person skilled in the art, for constructing the heterocycle A.

The compounds of the formula (IX) are known or can be synthesized by known processes from the appropriate starting materials.

During the reaction, the nitrogen of the amide in compounds of the formulae (II), (III), (IV), (V) and (VIII) may, if appropriate, be protected with a protective group known to the person skilled in the art, preferably a 2,4-dimethoxybenzyl group, which is removed under the conditions of the last step of the synthesis of the compounds of the formula (I).

The preparation of the compounds according to the invention can be illustrated by the synthesis schemes below:

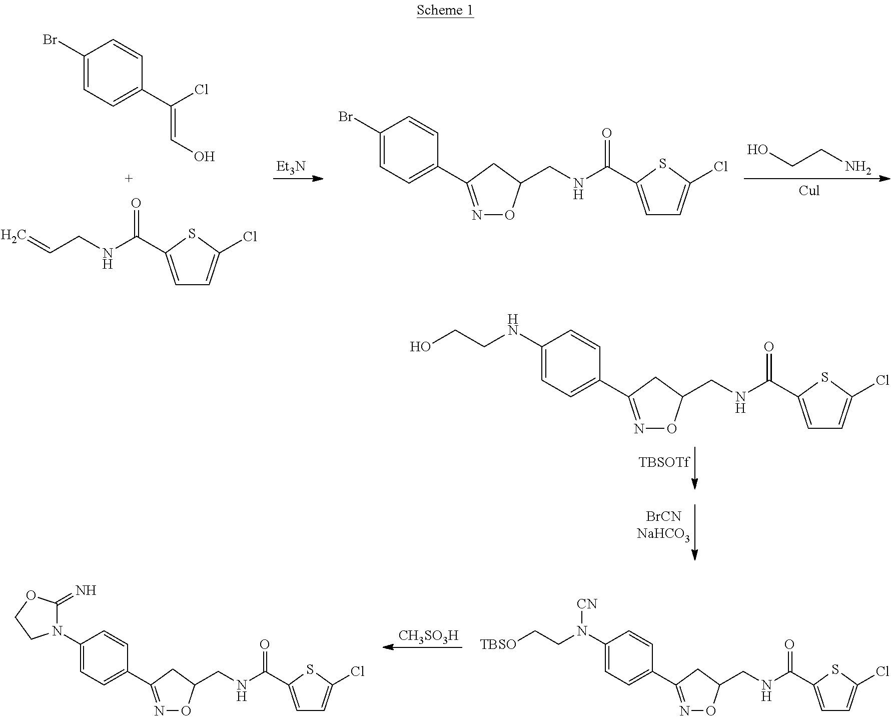

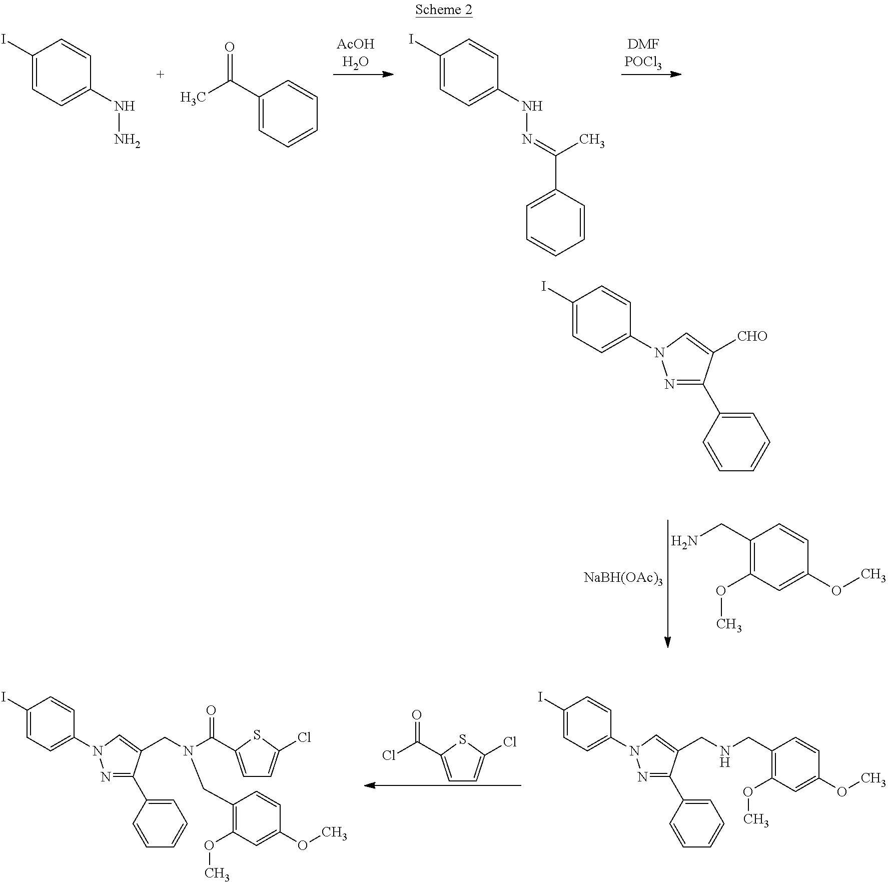

The compounds according to the invention have an unforeseeable useful pharmacological activity spectrum.

Accordingly, they are suitable for use as medicaments for the treatment and/or prophylaxis of diseases in humans and animals.

The compounds according to the invention are selective inhibitors of blood coagulation factor Xa which act in particular as anticoagulants.

In addition, the compounds according to the invention have favourable physicochemical properties, such as, for example, good solubility in water and physiological media, which is advantageous for their therapeutic application.

The present invention furthermore provides the use of the compounds according to the invention for the treatment and/or prophylaxis of disorders, preferably thromboembolic disorders and/or thromboembolic complications.

For the purposes of the present invention, “thromboembolic disorders” include in particular disorders such as ST-elevation myocardial infarction (STEMI) or non-ST-elevation myocardial infarction (non-STEMI), stable angina pectoris, unstable angina pectoris, reocclusions and restenoses after coronary interventions such as angioplasty or aortocoronary bypass, peripheral arterial occlusive diseases, pulmonary embolisms, deep vein thromboses and kidney vein thromboses, transitory ischaemic attacks and also thrombotic and thromboembolic stroke.

Accordingly, the substances are also suitable for preventing and treating cardiogenic thromboembolisms, such as, for example, brain ischaemias, stroke and systemic thromboembolisms and ischaemias, in patients having acute, intermittent or persistent cardioarrhythmias, such as, for example, atrial fibrillation, and those undergoing cardioversion, furthermore patients having heart valve disorders or having artificial heart valves. In addition, the compounds according to the invention are suitable for treating disseminated intravascular coagulation (DIC).

Thromboembolic complications furthermore occur during microangiopathic haemolytic anaemias, extracorporeal blood circulation, such as haemodialysis, and in connection with heart valve prostheses.

Moreover, the compounds according to the invention are also suitable for the prophylaxis and/or treatment of atherosclerotic vascular disorders and inflammatory disorders, such as rheumatic disorders of the locomotor apparatus, and in addition also for the prophylaxis and/or treatment of Alzheimer's disease. Moreover, the compounds according to the invention can be used for inhibiting tumour growth and formation of metastases, for microangiopathies, age-related macular degeneration, diabetic retinopathy, diabetic nephropathy and other microvascular disorders, and also for the prevention and treatment of thromboembolic complications, such as, for example, venous thromboembolisms, in tumour patients, in particular patients undergoing major surgical interventions or chemo- or radiotherapy.

The compounds according to the invention can additionally also be used for preventing coagulation ex vivo, for example for preserving blood and plasma products, for cleaning/pretreating catheters and other medical tools and instruments, for coating synthetic surfaces of medical tools and instruments used in vivo or ex vivo or for biological samples comprising factor Xa.

The present invention furthermore provides the use of the compounds according to the invention for the treatment and/or prophylaxis of disorders, in particular the disorders mentioned above.

The present invention furthermore provides the use of the compounds according to the invention for preparing a medicament for the treatment and/or prophylaxis of disorders, in particular the disorders mentioned above.

The present invention furthermore provides a method for the treatment and/or prophylaxis of disorders, in particular the disorders mentioned above, using an anticoagulatory effective amount of the compound according to the invention.

The present invention furthermore provides a method for preventing blood coagulation in vitro, in particular in banked blood or biological samples comprising factor Xa, which method is characterized in that an anticoagulatory effective amount of the compound according to the invention is added.

The present invention furthermore provides medicaments comprising a compound according to the invention and one or more further active compounds, in particular for the treatment and/or prophylaxis of the disorders mentioned above. The following compounds may be mentioned by way of example and by way of preference as active compounds suitable for combinations:
lipid-lowering agents, in particular HMG-CoA (3-hydroxy-3-methylglutaryl-coenzyme A) reductase inhibitors;
coronary therapeutics/vasodilators, in particular ACE (angiotensin converting enzyme) inhibitors; AII (angiotensin II) receptor antagonists; β-adrenoceptor antagonists; alpha-1-adrenoceptor antagonists; diuretics; calcium channel blockers; substances which cause an increase in the cyclic guanosine monophosphate (cGMP) concentration such as, for example, stimulators of soluble guanylate cyclase;
plasminogen activators (thrombolytics/fibrinolytics) and compounds enhancing thrombolysis/fibrinolysis, such as inhibitors of the plasminogen activator inhibitor (PAI inhibitors) or inhibitors of the thrombin-activated fibrinolysis inhibitor (TAFI inhibitors);
anticoagulants;
platelet aggregation inhibiting substances (platelet aggregation inhibitors, thrombocyte aggregation inhibitors);
fibrinogen receptor antagonists (glycoprotein-IIb/IIIa antagonists);
and also antiarrhythmics.

The present invention furthermore provides medicaments comprising at least one compound according to the invention, usually together with one or more inert non-toxic pharmaceutically acceptable auxiliaries, and their use for the purposes mentioned above.

The compounds according to the invention can act systemically and/or locally. For this purpose, they can be administered in a suitable way, such as, for example, by the oral, parenteral, pulmonary, nasal, sublingual, lingual, buccal, rectal, dermal, transdermal, conjunctival or otic route, or as implant or stent.

For these administration routes, it is possible to administer the compounds according to the invention in suitable administration forms.

Suitable for oral administration are administration forms which work as described in the prior art and deliver the compounds according to the invention rapidly and/or in modified form, which comprise the compounds according to the invention in crystalline and/or amorphous and/or dissolved form, such as, for example, tablets (uncoated and coated tablets, for example tablets provided with enteric coatings or coatings whose dissolution is delayed or which are insoluble and which control the release of the compound according to the invention), tablets which rapidly decompose in the oral cavity, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (for example intravenously, intraarterially, intracardially, intraspinally or intralumbarly) or with inclusion of absorption (for example intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates or sterile powders.

Examples suitable for other administration routes are pharmaceutical forms for inhalation (inter alia powder inhalers, nebulizers), nasal drops/solutions/sprays; tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Preference is given to oral or parenteral administration, in particular oral administration.

The compounds according to the invention can be converted into the stated administration forms. This can take place in a manner known per se by mixing with inert, non-toxic, pharmaceutically suitable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and flavour- and/or odour-masking agents.

In general, it has proved advantageous to administer on parenteral administration amounts of from about 0.001 to 1 mg/kg, preferably from about 0.01 to 0.5 mg/kg, of body weight to achieve effective results. The dosage on oral administration is from about 0.01 to 100 mg/kg, preferably about 0.01 to 20 mg/kg, and very particularly preferably 0.1 to 10 mg/kg, of body weight.

It may nevertheless be necessary, where appropriate, to deviate from the amounts mentioned, depending on the body weight, the administration route, the individual response to the active compound, the mode of preparation and the time or interval over which administration takes place. Thus, in some cases it may be sufficient to make do with less than the aforementioned minimal amount, whereas in other cases the upper limit mentioned must be exceeded. In the event of administration of larger amounts, it may be advisable to divide these into a plurality of individual doses over the day.

The invention is illustrated by the working examples below. The invention is not limited to the examples.

The percentage data in the following tests and examples are percentages by weight unless otherwise indicated; parts are parts by weight. Solvent ratios, dilution ratios and concentration data of liquid/liquid solutions are in each case based on volume.

### A. EXAMPLES

### Abbreviations

| | |
|---|---|
| TLC | Thin-Layer Chromatography |
| DCI | Direct Chemical Ionization (in MS) |
| DMF | N,N-Dimethylformamide |
| DMSO | Dimethyl sulphoxide |
| d | day(s) |
| ee | Enantiomeric excess |
| eq. | Equivalent(s) |
| ESI | Electrospray Ionization (in MS) |
| h | hour(s) |
| HPLC | High-Pressure, High-Performance Liquid Chromatography |
| LC-MS | Liquid Chromatography-coupled Mass Spectroscopy |
| min | minute(s) |
| MS | Mass Spectroscopy |
| NMR | Nuclear Magnetic Resonance spectroscopy |
| RP | Reversed Phase (in HPLC) |
| RT | Room Temperature |
| Rₜ | Retention time (in HPLC) |
| TBTU | O-(benzotriazol-1-yl)-N,N,N′,N′-tetramethyluronium |
| | tetrafluoroborate |
| THF | Tetrahydrofuran |

### LC-MS and HPLC Methods

Method 1: Instrument: HP 1100 with DAD detection; column: Kromasil 100 RP-18, 60 mm×2.1 mm, 3.5 μm; mobile phase A: 5 ml of perchloric acid (70% strength)/1 of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

Method 2: Instrument: HP 1100 with DAD detection; column: Kromasil 100 RP-18, 60 mm×2.1 mm, 3.5 μm; mobile phase A: 5 ml of perchloric acid (70% strength)/1 of water, mobile phase B: acetonitrile; gradient: 0 min 2% B→0.5 min 2% B→4.5 min 90% B→9 min 0% B→9.2 min 2% B→10 min 2% B; flow rate: 0.75 ml/min; column temperature: 30° C.; UV detection: 210 nm.

Method 3: MS instrument type: Micromass ZQ; HPLC instrument type: Waters Alliance 2795; Column: Phenomenex Synergi 2μ Hydro-RP Mercury 20 mm×4 mm; mobile phase A: 1 1 of water+0.5 ml of 50% strength formic acid, mobile phase B: 1 1 of acetonitrile+0.5 ml of 50% strength formic acid; Gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 4: MS instrument type: Micromass ZQ; HPLC instrument type: HP 1100 Series; UV DAD; column: Phenomenex Synergi 2μ Hydro-RP Mercury 20 mm×4 mm; mobile phase A: 1 1 of water+0.5 ml of 50% strength formic acid, mobile phase B: 1 1 of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 5: Instrument: Micromass Quattro LCZ with HPLC Agilent Series 1100; column: Phenomenex Synergi 2μ Hydro-RP Mercury 20 mm×4 mm; mobile phase A: 1 1 of water+0.5 ml of 50% strength formic acid, mobile phase B: 1 1 of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

Method 6: Column: GROM-SIL 120 ODS-4 HE, 10 μM, 250 mm×30 mm; mobile phase and gradient programme: acetonitrile/0.1% aqueous formic acid 10:90 (0-3 min), acetonitrile/0.1% aqueous formic acid 10:90→95:5 (3-27 min), acetonitrile/0.1% aqueous formic acid 95:5 (27-34 min), acetonitrile/0.1% aqueous formic acid 10:90 (34-38 min); flow rate: 50 ml/min; temperature: 22° C.; UV detection: 254 nm.

### Starting Materials

### Example 1A

### N-{[3-(4-Bromophenyl)-4,5-dihydroisoxazol-5-yl]methyl}-5-chlorothiophene-2-carboxamide

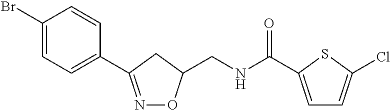

At 0° C., 594 μl (4.265 mmol) of triethylamine are added dropwise to a solution of 748 mg (3.708 mmol) of 4-bromo-N-hydroxybenzimidoyl chloride (M. R. Barbachyn et al., J. Med. Chem. 46(2), 284-302 (2003)) and 1.0 g (4.265 mmol) of N-allyl-5-chlorothiophenecarboxamide in 30 ml of anhydrous dichloromethane. The reaction mixture is stirred at room temperature for 15 hours. The mixture is then evaporated to dryness on a rotary evaporator. The residue obtained is triturated with a mixture of acetonitrile and water in a volume ratio of 1:1. The product insoluble therein is filtered off with suction, washed with acetonitrile and dried under high vacuum. This gives 1.1 g (71% of theory) of the title compound.

¹H-NMR (500 MHz, DMSO-d₆, δ/ppm): 8.90 (t, 1H), 7.67 (2 d, together 3H), 7.60 (d, 2H), 7.19 (d, 1H), 4.90-4.84 (m, 1H), 3.52 (dd, 1H), 3.45-3.42 (m, 2H), 3.21 (dd, 1H).

HPLC (Method 3): Rₜ=2.36 min.

MS (ESIpos, m/z): 399/401/403 (⁷⁹Br/⁸¹Br, ³⁵Cl/³⁷Cl) (M+H)⁺.

### Example 2A

### 5-Chloro-N-[(3-{4-[(2-hydroxyethyl)amino]phenyl}-4,5-dihydroisoxazol-5-yl)methyl]thiophene-2-carboxamide

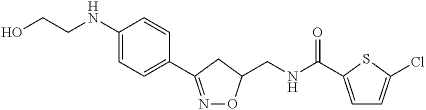

1.09 g (2.737 mmol) of the product from Example 1A are dissolved in 20 ml of anhydrous dioxane, and 397 μl (6.569 mmol) of aminoethanol, 104 mg (0.547 mmol) of copper(I) iodide, 2.32 g (10.95 mmol) of potassium phosphate and 175 μl (1.642 mmol) of N,N′-dimethylethylenediamine are added successively. The reflux apparatus is made inert by repeatedly applying a slight vacuum and venting with argon. The reaction mixture is heated at reflux for 15 hours. Since the conversion at this time is about 50%, the mixture is allowed to warm to RT, and the same amount of aminoethanol, copper(I) iodide, potassium phosphate and N,N′-dimethylethylenediamine are added again. After inertization, the mixture is heated at reflux for a further 20 hours. After this time, the mixture is allowed to cool to RT. Water is added, and the mixture is extracted with ethyl acetate. The organic extract is washed successively with water and saturated sodium chloride solution. The extract is dried over anhydrous magnesium sulphate and filtered, and the filtrate is freed from the solvent under reduced pressure. The residue is purified by preparative HPLC (method 6). The product fraction obtained is triturated with a mixture of acetonitrile and N,N-dimethylformamide. The solid is filtered off with suction, washed with acetonitrile and dried under high vacuum. This gives 152 mg (15% of theory) of the title compound.

¹H-NMR (500 MHz, DMSO-d₆, δ/ppm): 8.88 (t, 1H), 7.68 (d, 1H), 7.37 (d, 2H), 7.19 (d, 1H), 6.61 (d, 2H), 6.09 (t, 1H), 4.75-4.70 (m, 1H), 4.72 (t, 1H), 3.54 (dt, 2H), 3.42-3.35 (m, 3H), 3.17-3.08 (m, 3H).

HPLC (Method 1): Rₜ=3.62 min.

MS (DCI, NH₃, m/z): 380/382 (³⁵Cl/³⁷Cl) (M+H)⁺, 397/399 (M+NH₄)⁺.

### Example 3A

### N-[(3-{4-[(2-{[tert-Butyl(dimethyl)silyl]oxy}ethyl)amino]phenyl}-4,5-dihydroisoxazol-5-yl)methyl]-5-chlorothiophene-2-carboxamide

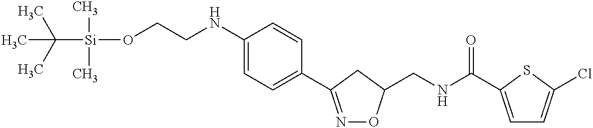

At −50° C., 93 μl (0.404 mmol) of tert-butyl(dimethyl)silyl trifluoromethanesulphonate are added to a suspension of 146 mg (0.384 mmol) of the product from Example 2A and 67 μl (0.577 mmol) of 2,6-dimethylpyridine in 15 ml of anhydrous dichloromethane. The reaction mixture is stirred at room temperature for 15 hours. About 30 ml of water are then added, and the mixture is extracted with dichloromethane. The extract is washed with water, dried over anhydrous sodium sulphate, filtered and freed from the solvent on a rotary evaporator. The residue is purified by preparative HPLC (Method 6). This gives 136 mg (72% of theory) of the title compound.

¹H-NMR (500 MHz, DMSO-d₆, δ/ppm): 8.86 (t, 1H), 7.66 (d, 1H), 7.33 (d, 2H), 7.17 (d, 1H), 6.58 (d, 2H), 6.08 (t, 1H), 4.73-4.67 (m, 1H), 3.67 (t, 2H), 3.39-3.33 (m, 3H), 3.17 (dt, 2H), 3.08 (dd, 1H), 0.83 (s, 9H), 0.01 (s, 6H).

HPLC (Method 3): Rₜ=2.99 min.

MS (ESIpos, m/z): 494/496 (³⁵Cl/³⁷Cl) (M+H)⁺.

### Example 4A

### N-[(3-{4-[(2-{[tert-Butyl(dimethyl)silyl]oxy}ethyl)(cyano)amino]phenyl}-4,5-dihydroisoxazol-5-yl)methyl]-5-chlorothiophene-2-carboxamide

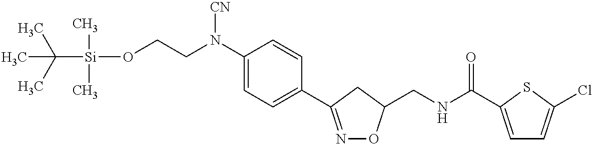

In a thick-walled glass tube with screw-on lid, a mixture of 106 mg (0.215 mmol) of the product from Example 3A, 54 mg (0.644 mmol) of sodium bicarbonate and 86 μl (0.257 mmol) of a 3 molar solution of cyanogen bromide in dichloromethane in 5 ml of tetrahydrofuran is heated at 40-50° C. for a total of 5 days. After each of days one to four, the reaction vessel is opened at room temperature, and the same amounts of cyanogen bromide solution and sodium bicarbonate are added again. After day five, the reaction mixture is diluted with dichloromethane and washed successively with water, saturated sodium bicarbonate solution and saturated sodium chloride solution. After drying over anhydrous sodium sulphate, filtration and removal of the solvent on a rotary evaporator, the residue is dissolved in acetonitrile and the same volume of water is added. This results in the product precipitating out. The product is filtered off with suction and dried under high vacuum. This gives 71 mg (64% of theory) of the title compound.

¹H-NMR (400 MHz, DMSO-d₆, δ/ppm): 8.87 (t, 1H), 7.68 (d, 2H), 7.67 (d, 1H), 7.25 (d, 2H), 7.17 (d, 1H), 4.87-4.81 (m, 1H), 3.88-3.83 (m, 4H), 3.48 (dd, 1H), 3.45-3.40 (m, 2H), 3.18 (dd, 1H), 0.82 (s, 9H), −0.05 (s, 6H).

HPLC (Method 2): Rₜ=5.33 min.

MS (DCI, NH₃, m/z): 519/521 (³⁵Cl/³⁷Cl) (M+H)⁺, 536/538 (M+NH₄)⁺.

### Example 5A

### Acetophenone-(4-iodophenyl)hydrazone

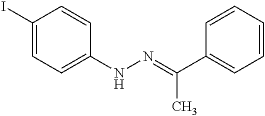

A solution of 1.54 g (12.82 mmol) of acetophenone in 10 ml of the same solvent is added to a solution of 2.0 g (8.546 mmol) of 4-iodophenylhydrazine in 30 ml of 50% strength acetic acid. The mixture is stirred at room temperature, and a precipitate is formed. After 30 minutes, the precipitate is filtered off and washed thoroughly first with water and then with cyclohexane. The residue is dried under high vacuum. This gives 1.95 g (68% of theory) of the title compound.

¹H-NMR (400 MHz, DMSO-d₆, δ/ppm): 7.78 (d, 2H), 7.51 (d, 2H), 7.38 (dd, 2H), 7.30 (dd, 1H), 7.07 (d, 2H), 2.25 (s, 3H).

HPLC (Method 4): Rₜ=3.22 min.

MS (ESIpos, m/z): 337 (M+H)⁺.

### Example 6A

### 1-(4-Iodophenyl)-3-phenyl-1H-pyrazole-4-carbaldehyde

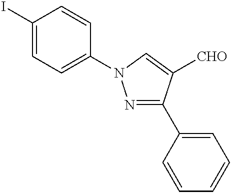

At 0° C., 1.08 ml (11.58 mmol) of phosphoryl chloride (POCl₃) are slowly added dropwise to 10 ml of anhydrous N,N-dimethylformamide. After 30 minutes at 0° C., a solution of 1.95 g (5.792 mmol) of the product from Example 5A in 10 ml of N,N-dimethylformamide is added dropwise, and the reaction mixture is stirred at 0° C. for a further hour. The mixture is then allowed to warm to room temperature, stirred for a further hour and then warmed to 60° C. The reaction mixture is stirred at this temperature for 15 hours. The mixture is then allowed to cool to room temperature, 80 ml of saturated sodium bicarbonate solution are added and the mixture is extracted with ethyl acetate. The organic extract is washed successively with water and saturated sodium chloride solution. After drying over anhydrous sodium sulphate, the extract is filtered and the solvent is removed on a rotary evaporator. The residue obtained is triturated with diisopropyl ether. The solid is filtered off with suction, washed with diisopropyl ether and dried under high vacuum. This gives 1.34 g (62% of theory) of the title compound.

¹H-NMR (400 MHz, DMSO-d₆, δ/ppm): 9.98 (s, 1H), 9.36 (s, 1H), 7.95-7.90 (m, 4H), 7.82 (d, 2H), 7.53-7.48 (m, 3H).

HPLC (Method 4): Rₜ=3.08 min.

MS (ESIpos, m/z): 375 (M+H)⁺.

### Example 7A

### 1-(2,4-Dimethoxyphenyl)-N-{[1-(4-iodophenyl)-3-phenyl-1H-pyrazol-4-yl]methyl}methaneamine

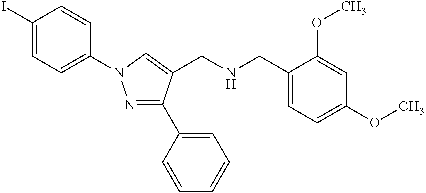

1.34 g (3.581 mmol) of the product from Example 6A and 538 μl (3.581 mmol) of 2,4-dimethoxybenzylamine are dissolved in 40 ml of dichloroethane and stirred at room temperature for one hour. 1.52 g (7.162 mmol) of sodium triacetoxyborohydride and 820 μl (14.33 mmol) of glacial acetic acid are then added. The reaction mixture is stirred at room temperature for 15 hours. A saturated sodium bicarbonate solution is then added, and the product is extracted with dichloromethane. The organic extract is washed with water and dried over anhydrous sodium sulphate. After filtration, the solvent is removed on a rotary evaporator. The crude product is dried under high vacuum and used for the next reaction without further purification. 1.89 g of the title compound are obtained.

HPLC (Method 5): Rₜ=2.10 min (60%).

MS (ESIpos, m/z): 526 (M+H)⁺.

### Example 8A

### 5-Chloro-N-(2,4-dimethoxybenzyl)-N-{[1-(4-iodophenyl)-3-phenyl-1H-pyrazol-4-yl]methyl}thiophene-2-carboxamide

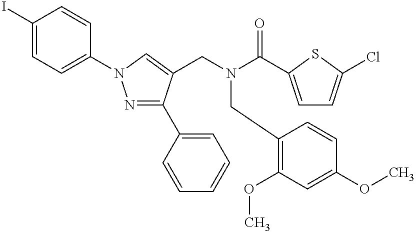

A solution of 651 mg (3.597 mmol) of 5-chlorothiophene-2-carbonyl chloride in 10 ml anhydrous tetrahydrofuran is added with a solution of 1.89 g (3.597 mmol) of the product from Example 7A and 1.25 ml of diisopropylethylamine (Hünig base) in 40 ml of anhydrous tetrahydrofuran. The reaction mixture is stirred at room temperature for 15 hours. The solvent is then removed on a rotary evaporator and the residue is taken up in dichloromethane and washed successively with saturated sodium bicarbonate solution and water. After drying over anhydrous sodium sulphate, the mixture is filtered and evaporated and the residue is purified by preparative HPLC (method 6). This gives 1.05 g (43% of theory) of the title compound.

¹H-NMR (500 MHz, DMSO-d₆, δ/ppm): 8.53 (broad, 1H), 7.85 (d, 2H), 7.76 (d, 2H), 7.61 (d, 2H), 7.43-7.38 (m, 3H), 7.15 (broad, 1H), 7.08 (d, 1H), 7.01 (broad, 1H), 6.48-6.43 (m, 2H), 4.70 (broad, 2H), 4.58 (s, broad, 2H), 3.71 (s, 3H), 3.57 (broad, 3H).

HPLC (Method 2): Rₜ=6.47 min.

MS (ESIpos, m/z): 670/672 (³⁵Cl/³⁷Cl) (M+H)⁺.

### Example 9A

### 5-Chloro-N-(2,4-dimethoxybenzyl)-N-[(1-{4-[(2-hydroxyethyl)amino]phenyl}-3-phenyl-1H-pyrazol-4-yl)methyl]thiophene-2-carboxamide

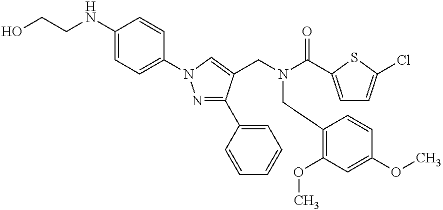

372 mg (0.555 mmol) of the compound from Example 8A are reacted with aminoethanol as described in Example 2A. Purification by preparative HPLC (method 6) gives 158 mg (47% of theory) of the title compound.

¹H-NMR (400 MHz, DMSO-d₆, δ/ppm): 8.18 (s, broad, 1H), 7.57-7.54 (m, 4H), 7.41-7.32 (m, 3H), 7.16 (broad, 1H), 7.07 (d, 1H), 7.00 (broad, 1H), 6.68 (d, 2H), 6.49-6.43 (m, 2H), 5.73 (t, 1H), 4.72-4.67 (m, 2H), 4.56 (s, broad, 2H), 3.72 (s, 3H), 3.60-3.54 (m, 5H), 3.13 (dt, 2H).

HPLC (Method 1): Rₜ=4.74 min.

MS (ESIpos, m/z): 603/605 (³⁵Cl/³⁷Cl) (M+H)⁺.

### Example 10A

### N-[(1-{4-[(2-{[tert-Butyl(dimethyl)silyl]oxy}ethyl)amino]phenyl}-3-phenyl-1H-pyrazol-4-yl)methyl]-5-chloro-N-(2,4-dimethoxybenzyl)thiophene-2-carboxamide

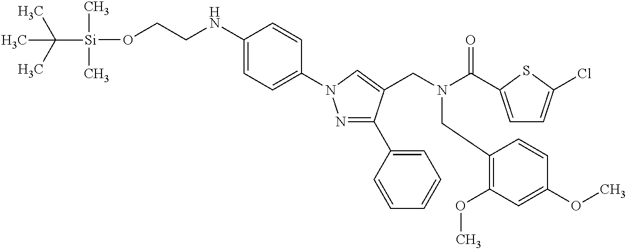

210 mg (0.350 mmol) of the compound from Example 9A are reacted analogously to the process described in Example 3A to give 194 mg (78% of theory) of the title compound.

¹H-NMR (500 MHz, DMSO-d₆, δ/ppm): 8.20 (s, broad, 1H), 7.57-7.54 (m, 4H), 7.41-7.32 (m, 3H), 7.15 (broad, 1H), 7.08 (d, 1H), 7.00 (broad, 1H), 6.68 (d, 2H), 6.49-6.43 (m, 2H), 5.77 (t, 1H), 4.70 (broad, 2H), 4.56 (s, broad, 2H), 3.73 (t, 2H), 3.71 (s, 3H), 3.58 (broad, 3H), 3.19 (dt, 2H).

HPLC (Method 2): Rₜ=5.86 min.

MS (ESIpos, m/z): 717/719 (³⁵Cl/³⁷Cl) (M+H)⁺.

### Example 11A

### N-[(1-{4-[(2-{[tert-Butyl(dimethyl)silyl]oxy}ethyl)(cyano)amino]phenyl}-3-phenyl-1H-pyrazol-4-yl)methyl]-5-chloro-N-(2,4-dimethoxybenzyl)thiophene-2-carboxamide

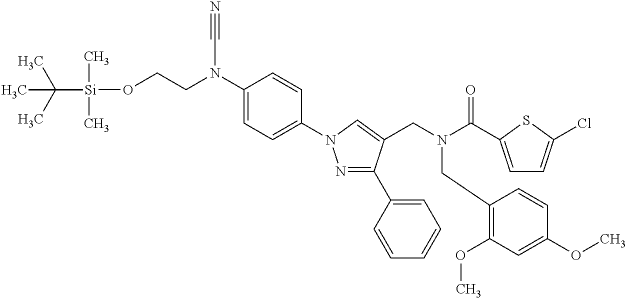

192 mg (0.268 mmol) of the compound from Example 10A are reacted analogously to the process described in Example 4A to give 173 mg (87% of theory) of the title compound.

¹H-NMR (500 MHz, DMSO-d₆, δ/ppm): 8.47 (broad, 1H), 7.97 (d, 2H), 7.61 (d, 2H), 7.46-7.38 (m, 3H), 7.33 (d, 2H), 7.17 (broad, 1H), 7.10 (d, 1H), 7.03 (broad, 1H), 6.50-6.45 (m, 2H), 4.73 (broad, 2H), 4.60 (s, broad, 2H), 3.91-3.87 (m, 4H), 3.73 (s, 3H), 3.59 (broad, 3H).

HPLC (Method 3): Rₜ=3.41 min.

MS (ESIpos, m/z): 742/744 (³⁵Cl/³⁷Cl) (M+H)⁺.

### Example 12A

### 1,1,1-Trifluoroacetone-(4-iodophenyl)hydrazone

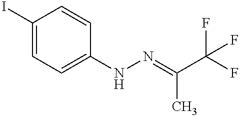

Analogously to the process described in Example 5A, 2.5 g (10.68 mmol) of 4-iodophenylhydrazone and 2.28 ml (16.02 mmol) of trifluoroacetone are reacted to give 2.18 g (62% of theory) of the title compound.

¹H-NMR (400 MHz, DMSO-d₆, δ/ppm): 7.57 (d, 2H), 7.01 (d, 2H), 2.05 (s, 3H).

HPLC (Method 3): Rₜ=2.74 min.

MS (ESIneg, m/z): 327 (M−H)⁺.

### Example 13A

### 1-(4-Iodophenyl)-3-(trifluoromethyl)-1H-pyrazole-4-carbaldehyde

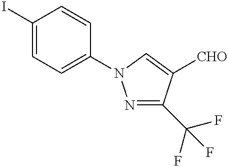

Analogously to the process described under Example 6A, 2.18 g (6.64 mmol) of the compound from Example 12A are converted into 2.46 g (100% of theory) of the title compound.

¹H-NMR (400 MHz, DMSO-d₆, δ/ppm): 9.97 (s, 1H), 9.50 (s, 1H), 7.97 (d, 2H), 7.50 (d, 2H).

HPLC (Method 4): Rₜ=2.89 min.

### Example 14A

### 1-(2,4-Dimethoxyphenyl)-N-{[1-(4-iodophenyl)-3-(trifluoromethyl)-1H-pyrazol-4-yl]methyl}methanamine

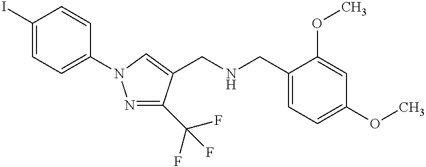

Analogously to the process described in Example 7A, 2.43 g (6.64 mmol) of the compound from Example 13A are converted into 3.46 g (100% of theory) of the title compound.

HPLC (Method 3): Rₜ=1.87 min.

MS (ESIpos, m/z): 518 (M+H)⁺.

### Example 15A

### 5-Chloro-N-(2,4-dimethoxybenzyl)-N-{[1-(4-iodophenyl)-3-(trifluoromethyl)-1H-pyrazol-4-yl]methyl}thiophene-2-carboxamide

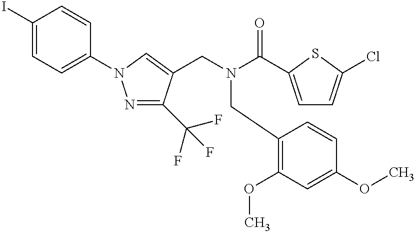

Analogously to the process described in Example 8A, 3.43 g (6.64 mmol) of the compound from Example 14A are converted into 987 mg (22% of theory) of the title compound

¹H-NMR (400 MHz, DMSO-d₆, δ/ppm): 8.63 (s, broad, 1H), 7.90 (d, 2H), 7.71 (d, 2H), 7.21 (d, 2H), 7.11 (d, 1H), 7.09 (broad, 1H), 6.54-6.49 (m, 2H), 4.68 (s, broad, 2H), 4.58 (s, broad, 2H), 3.73 (s, 3H), 3.70 (s, 3H).

HPLC (Method 2): Rₜ=6.25 min.

### Example 16A

### Acetaldehyde-(4-iodophenyl)hydrazone

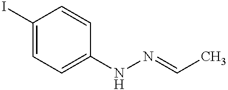

Analogously to the process described in Example 5A, 17.5 g (74.77 mmol) of 4-iodophenylhydrazone and 6.27 ml (112.2 mmol) of acetaldehyde are converted into 12.5 g (64% of theory) of the title compound.

¹H-NMR (400 MHz, DMSO-d₆, δ/ppm): 9.18 (s, broad, 1H), 7.46 (d, 2H), 6.91 (d, 2H), 6.57 (quart, 1H), 1.83 (d, 3H).

HPLC (Method 1): Rₜ=4.59 min.

MS (ESIpos, m/z): 261 (M+H)⁺.

### Example 17A

### 1-(4-Iodophenyl)-1H-pyrazole-4-carbaldehyde

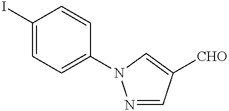

Analogously to the process described in Example 6A, 12.5 g (48.06 mmol) of the compound from Example 16A are converted into 6.37 g (40% of theory, based on a purity of 90%) of the title compound. Instead of 60° C., the mixture is stirred at 80° C.

¹H-NMR (400 MHz, DMSO-d₆, δ/ppm): 9.91 (s, 1H), 9.27 (s, 1H), 8.29 (s, 1H), 7.91 (d, 2H), 7.73 (d, 2H).

HPLC (Method 1): Rₜ=4.44 min.

MS (ESIpos, m/z): 299 (M+H)⁺.

### Example 18A

### 1-(2,4-Dimethoxyphenyl)-N-{[1-(4-iodophenyl)-1H-pyrazol-4-yl]methyl}methanamine

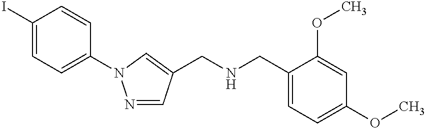

Analogously to the process described in Example 7A, 6.30 g (21.14 mmol) of the compound from Example 17A are converted into 9.5 g (62% of theory, based on a purity of 62%) of the title compound.

HPLC (Method 5): Rₜ=1.70 min.

MS (ESIpos, m/z): 450 (M+H)⁺.

### Example 19A

### 5-Chloro-N-(2,4-dimethoxybenzyl)-N-{[1-(4-iodophenyl)-1H-pyrazol-4-yl]methyl}thiophene-2-carboxamide

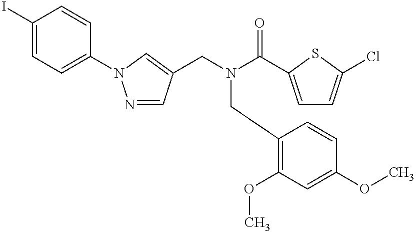

Analogously to the process described in Example 8A, 9.5 g (21.13 mmol) of the compound from Example 18A are converted into 5.14 g (37% of theory) of the title compound.

¹H-NMR (500 MHz, DMSO-d₆, δ/ppm): 8.33 (broad, 1H), 7.82 (d, 2H), 7.63-7.60 (m, 3H), 7.17 (broad, 1H), 7.11-7.10 (m, 2H), 6.53-6.51 (m, 2H), 4.62 (broad, 2H), 4.46 (broad, 2H), 3.73 (s, 3H), 3.71 (s, 3H).

HPLC (Method 3): Rₜ=3.08 min.

MS (ESIpos, m/z): 594/596 (³⁵Cl/³⁷Cl) (M+H)⁺.

### WORKING EXAMPLES

### Example 1

### 5-Chloro-N-({3-[4-(2-imino-1,3-oxazolidin-3-yl)phenyl]-4,5-dihydroisoxazol-5-yl}methyl)thiophene-2-carboxamide

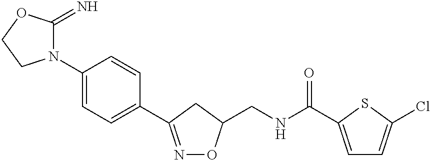

At room temperature, a suspension of 71 mg (0.137 mmol) of the compound from Example 4A and 19 μl (0.287 mmol) of methanesulphonic acid in 10 ml of anhydrous acetonitrile is stirred for 15 hours. A clear solution is formed, which is evaporated to dryness on a rotary evaporator. The residue is taken up into 2 ml of water, and 0.6 ml of saturated sodium bicarbonate solution is added. This results in the precipitation of the product. The solid is filtered off with suction, washed with water and dried under high vacuum. This gives 48 mg (87% of theory) of the title compound.

¹H-NMR (500 MHz, DMSO-d₆, δ/ppm): 8.90 (t, 1H), 7.90 (d, 2H), 7.68 (d, 1H), 7.62 (d, 2H), 7.19 (d, 1H), 6.33 (s, broad, 1H), 4.85-4.79 (m, 1H), 4.35 (t, 2H), 4.02 (t, 2H), 3.49 (dd, 1H), 3.43-3.40 (m, 2H), 3.20 (dd, 1H).

HPLC (Method 1): Rₜ=3.75 min.

MS (ESIpos, m/z): 405/407 (³⁵Cl/³⁷Cl) (M+H)⁺.

### Example 2

### 5-Chloro-N-({1-[4-(2-imino-1,3-oxazolidin-3-yl)phenyl]-3-phenyl-1H-pyrazol-4-yl}methyl)thiophene-2-carboxamide hydrochloride

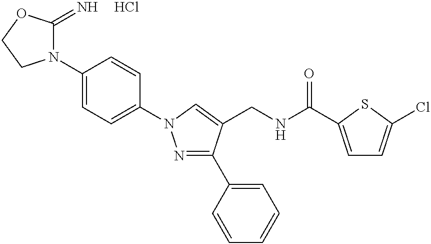

172 mg (0.232 mmol) of the compound from Example 11A are reacted analogously to the process described in Example 1. However, 0.5 ml of trifluoroacetic acid is added to the reaction mixture prior to evaporation to dryness, and the mixture is stirred at 40° C. for 30 minutes. All volatile components are then removed on a rotary evaporator. The residue obtained is taken up in a little acetonitrile and filtered through Celite. The filtrate is concentrated and dissolved in methanol, and 1 ml of 1 molar hydrochloric acid is added. The mixture is concentrated again. Dissolution in methanol and evaporation after addition of hydrochloric acid is repeated once more. This gives 110 mg (88% of theory) of the title compound.

¹H-NMR (500 MHz, DMSO-d₆, δ/ppm): 9.64 (s, broad, 1H), 9.07 (t, 1H), 8.90 (s, broad, 1H), 8.57 (s, 1H), 8.09 (d, 2H), 7.77 (d, 2H), 7.69 (d, 1H), 7.66 (d, 2H), 7.51-7.48 (m, 2H), 7.43-7.41 (m, 1H), 7.19 (d, 1H), 4.77 (t, 2H), 4.53 (d, 2H), 4.27 (t, 2H).

HPLC (Method 1): Rₜ=4.32 min.

MS (ESIpos, m/z): 478/480 (³⁵Cl/³⁷Cl) (M+H)⁺.

### Example 3

### 5-Chloro-N-({1-[4-(2-imino-1,3-oxazolidin-3-yl)phenyl]-3-(trifluoromethyl)-1H-pyrazol-4-yl}methyl)thiophene-2-carboxamide hydrochloride

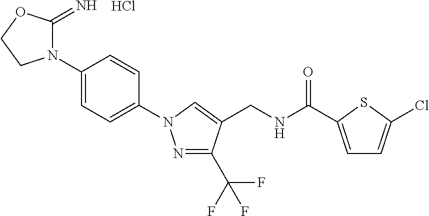

The title compound is obtained from the compound from Example 15A analogously to the processes described in Examples 9A, 10A, 11A and 2.

### Example 4

### 5-Chloro-N-({1-[4-(2-imino-1,3-oxazolidin-3-yl)phenyl]-1H-pyrazol-4-yl}methyl)thiophene-2-carboxamide hydrochloride

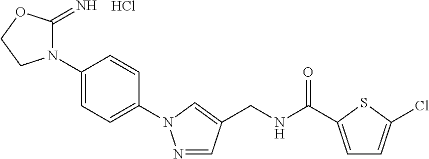

The title compound is obtained from the compound from Example 19A analogously to the processes described in Examples 9A, 10A, 11A and 2.

### B. EVALUATION OF THE PHARMACOLOGICAL ACTIVITY

The compounds according to the invention act in particular as selective inhibitors of blood coagulation factor Xa and do not, or only at significantly higher concentrations, inhibit other serine proteases, such as plasmin or trypsin.

“Selective” are those inhibitors of the blood coagulation factor Xa in which the IC₅₀ values for the factor Xa inhibition are lower by a factor of at least 100 compared to the IC₅₀ values for the inhibition of other serine proteases, in particular plasmin and trypsin, where, with respect to the test methods for the selectivity, reference is made to the test methods, described below, of Examples B.a.1) and B.a.2).

The advantageous pharmacological properties of the compounds according to the invention can be determined by the following methods:

### a) Test Descriptions (In Vitro)

### a.1) Determination of the Factor Xa Inhibition

In order to determine the factor Xa inhibition of the substances listed above, a biochemical test system is set up, in which the conversion of a factor Xa substrate is used to determine the enzymatic activity of human factor Xa. Factor Xa cleaves aminomethylcoumarin, whose fluorescence is measured, from the peptidic substrate. The determinations are carried out in microtitre plates.

Substances to be tested, in various concentrations, are dissolved in dimethyl sulphoxide and incubated for 15 min at 22° C. with human Factor Xa (1.3 mmol/l dissolved in 50 mmol/l of Tris buffer [C,C,C-tris(hydroxymethyl)aminomethane], 100 mmol/l NaCl, 0.1% BSA [bovine serum albumin], pH 7.4). The substrate (5 μmol/l of Boc-Ile-Glu-Gly-Arg-AMC from Bachem) is then added. After an incubation time of 30 min, the sample is excited at a wavelength of 360 nm, and the emission at 460 nm is measured. The measured emissions of the test batches with test substance are compared to the control batches without test substance (only dimethyl sulphoxide instead of test substance in dimethyl sulphoxide), and IC₅₀ values are calculated from the concentration/activity relationships.

Representative activity data from this test are listed in Table 1 below:

TABLE 1

| Example No. | IC₅₀ [nM] |
|---|---|
| 1 | 7.9 |
| 2 | 1.4 |

### a.2) Determination of the Selectivity

To demonstrate the selectivity of the substances with respect to factor Xa inhibition, the test substances are examined for their inhibition of other human serine proteases, such as trypsin and plasmin. To determine the enzymatic activity of trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of NaCl, 0.1% BSA [bovine serum albumin], 5 mmol/l of calcium chloride, pH 7.4) and incubated for 15 min with various concentrations of test substance in dimethyl sulphoxide and also with dimethyl sulphoxide without test substance. The enzymatic reaction is then started by addition of the appropriate substrates (5 μmol/l of Boc-Ile-Glu-Gly-Arg-AMC from Bachem for trypsin and 50 μmol/l of MeOSuc-Ala-Phe-Lys-AMC from Bachem for plasmin). After an incubation time of 30 min at 22° C., the fluorescence is measured (excitation: 360 nm, emission: 460 nm). The measured emissions of the test batches with test substance are compared to the control batches without test substance (only dimethyl sulphoxide instead of test substance in dimethyl sulphoxide), and IC₅₀ values are calculated from the concentration/activity relationships.

### a.3) Determination of the Anticoagulatory Activity

The anticoagulatory activity of the test substances is determined in vitro in human and rabbit plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 2500 g for 10 minutes. The supernatant is pipetted off. The prothrombin time (PT, synonyms: thromboplastin time, quick test) is determined in the presence of varying concentrations of test substance or the corresponding solvent using a commercial test kit (Hemoliance® RecombiPlastin, from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

### b) Determination of the Antithrombotic Activity (In Vivo)

### b.1) Arteriovenous Shunt Model (Rabbit)

Fasting rabbits (strain: Esd: NZW) are anaesthetized by intramuscular administration of Rompun/Ketavet solution (5 mg/kg and 40 mg/kg, respectively). Thrombus formation is initiated in an arteriovenous shunt in accordance with the method described by C. N. Berry et al. [Semin. Thromb. Hemost. 1996, 22, 233-241]. To this end, the left jugular vein and the right carotid artery are exposed. The two vessels are connected by an extracorporeal shunt using a vein catheter of a length of 10 cm. In the middle, this catheter is attached to a further polyethylene tube (PE 160, Becton Dickenson) of a length of 4 cm which contains a roughened nylon thread which has been arranged to form a loop, to form a thrombogenic surface. The extracorporeal circulation is maintained for 15 minutes. The shunt is then removed and the nylon thread with the thrombus is weighed immediately. The weight of the nylon thread on its own was determined before the experiment was started. Before extracorporeal circulation is set up, the test substances are administered either intravenously via an ear vein or orally using a pharyngeal tube.

### c) Solubility Assay

### Reagents Required:

PBS buffer pH 7.4: 90.00 g of NaCl p.a. (for example Merck Art. No. 1.06404.1000), 13.61 g of KH₂PO₄ p.a. (for example Merck Art. No. 1.04873.1000) and 83.35 g of 1N NaOH (for example Bernd Kraft GmbH Art. No. 01030.4000) are weighed into a 1 1 measuring flask, the flask is filled with water and the mixture is stirred for about 1 hour.
Acetate buffer pH 4.6: 5.4 g of sodium acetate×3H₂O p.a. (for example Merck Art. No. 1.06267.0500) are weighed into a 100 ml measuring flask and dissolved in 50 ml of water, 2.4 g of glacial acetic acid are added, the mixture is made up to 100 ml with water, the pH is checked and, if required, adjusted to pH 4.6.
Dimethyl sulphoxide (for example Baker Art. No. 7157.2500)
Distilled water

### Preparation of the Calibration Solutions:

Preparation of the stock solution of calibration solutions: About 0.5 mg of the active compound are weighed accurately into a 2 ml Eppendorf Safe-Lock tube (Eppendorf Art. No. 0030 120.094), DMSO is added to a concentration of 600 μg/ml (for example 0.5 mg of active compound+833 μl of DMSO) and the mixture is vortexed until everything has gone into solution.

Calibration solution 1 (20 μg/ml): 1000 μl of DMSO are added to 34.4 μl of the stock solution, and the mixture is homogenized.

Calibration solution 2 (2.5 μg/ml): 700 μl of DMSO are added to 100 μl of calibration solution 1, and the mixture is homogenized.

### Preparation of the Sample Solutions:

Sample solution for solubilities of up to 10 g/l in PBS buffer pH 7.4: About 5 mg of the active compound are weighed accurately into a 2 ml Eppendorf Safe-Lock tube (Eppendorf Art. No. 0030 120.094), and PBS buffer pH 7.4 is added to a concentration of 5 g/l (for example 5 mg of active compound+500 μl of PBS buffer pH 7.4).

Sample solution for solubilities of up to 10 g/l in acetate buffer pH 4.6: About 5 mg of the active compound are weighed accurately into a 2 ml Eppendorf Safe-Lock tube (Eppendorf Art. No. 0030 120.094), and acetate buffer pH 4.6 is added to a concentration of 5 g/l (for example 5 mg of active compound+500 μl of acetate buffer pH 4.6).

Sample solution for solubilities of up to 10 g/l in water: About 5 mg of the active compound are weighed accurately into a 2 ml Eppendorf Safe-Lock tube (Eppendorf Art. No. 0030 120.094), and water is added to a concentration of 5 g/l (for example 5 mg of active compound+500 μl of water).

### Practice:

The sample solutions prepared in this manner are shaken at 1400 rpm in a temperature-adjustable shaker (for example Eppendorf Thermomixer comfort Art. No. 5355 000.011 with interchangeable block Art. No. 5362.000.019) at 20° C. for 24 hours. In each case 180 μl are taken from these solutions and transferred into Beckman Polyallomer centrifuge tubes (Art. No. 343621). These solutions are centrifuged at about 223 000 *g for 1 hour (for example Beckman Optima L-90K ultracentrifuge with type 42.2 Ti rotor at 42 000 rpm). From each of the sample solutions, 100 μl of the supernatant are removed and diluted 1:5, 1:100 and 1:1000 with the respective solvent used (water, PBS buffer 7.4 or acetate buffer pH 4.6). From each dilution, a sample is transferred into a vessel suitable for HPLC analysis.

### Analysis:

The samples are analyzed by RP-HPLC. Quantification is carried out using a two-point calibration curve of the test compound in DMSO. The solubility is expressed in mg/l.

### Analysis Sequence:

1. Calibration solution 2.5 mg/ml
2. Calibration solution 20 μg/ml
3. Sample solution 1:5
4. Sample solution 1:100
5. Sample solution 1:1000

### HPLC Method for Acids:

Agilent 1100 with DAD (G1315A), quat. pump (G1311A), autosampler CTC HTS PAL, degasser (G1322A) and column thermostat (G1316A); column: Phenomenex Gemini C18, 50×2 mm, 5μ; temperature: 40° C.; mobile phase A: water/phosphoric acid pH 2; mobile phase B: acetonitrile; flow rate: 0.7 ml/min; gradient: 0-0.5 min 85% A, 15% B; ramp: 0.5-3 min 10% A, 90% B; 3-3.5 min 10% A, 90% B; ramp: 3.5-4 min 85% A, 15% B; 4-5 min 85% A, 15% B.

### HPLC Method for Bases:

Agilent 1100 with DAD (G1315A), quat. pump (G1311A), autosampler CTC HTS PAL, degasser (G1322A) and column thermostat (G1316A); column: VDSoptilab Kromasil 100 C18, 60×2.1 mm, 3.5μ; temperature: 30° C.; mobile phase A: water+5 ml perchloric acid/1; mobile phase B: acetonitrile; flow rate: 0.75 ml/min; gradient: 0-0.5 min 98% A, 2% B; ramp: 0.5-4.5 min 10% A, 90% B; 4.5-6 min 10% A, 90% B; ramp: 6.5-6.7 min 98% A, 2% B; 6.7-7.5 min 98% A, 2% B.

### C. EXEMPLARY EMBODIMENTS OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Preparation:

The mixture of the compound according to the invention, lactose and starch is granulated with a 5% strength solution (m/m) of PVP in water. The granules are dried and then mixed with the magnesium stearate for 5 minutes. This mixture is compressed using a conventional tablet press (see above for format of the tablet). As guideline, a compressive force of 15 kN is used for the compression.

### Oral Suspension:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension are equivalent to a single dose of 100 mg of the compound according to the invention.

### Preparation:

The Rhodigel is suspended in ethanol, and the compound according to the invention is added to the suspension. The water is added while stirring. The mixture is stirred for about 6 h until the swelling of the Rhodigel is complete.

### Oral Solution:

### Composition:

500 mg of the compound according to the invention, 2.5 g of polysorbate and 97 g of polyethylene glycol 400.20 g of oral solution are equivalent to a single dose of 100 mg of the compound according to the invention.

### Production:

The compound according to the invention is suspended in the mixture of polyethylene glycol and polysorbate while stirring. Stirring is continued until the compound according to the invention is completely dissolved.

I.v. Solution:

The compound according to the invention is dissolved at a concentration below saturation solubility in a physiologically acceptable solvent (for example isotonic sodium chloride solution, glucose solution 5% and/or PEG 400 solution 30%). The solution is sterilized by filtration and filled into sterile and pyrogen-free injection containers.

1. Compound of the formula 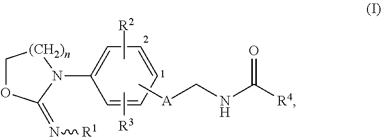
in which n represents the number 1, 2 or 3
A represents a 5-membered heteroaryl or a 5-membered heterocyclyl, where heteroaryl and heterocyclyl are attached in the 1- or 2-position to the phenyl ring and heteroaryl and heterocyclyl for their part have a 1,3-attachment to the phenyl ring and the carbonylaminomethyl group, and where heteroaryl and heterocyclyl may be substituted by a substituent R⁸, where R⁸ is attached to the neighbouring atom of the atom to which the carbonylaminomethyl group is attached and has a 1,4-attachment to the phenyl ring and where the atom to which R⁸ is attached is a nitrogen or carbon atom and where R⁸ represents halogen, hydroxy, amino, C₁-C₄-alkyl, C₁-C₄-alkoxy, C₁-C₄-alkylamino, hydroxycarbonyl, aminocarbonyl, C₁-C₄-alkoxycarbonyl, C₁-C₄-alkylaminocarbonyl, aminosulphonyl, C₁-C₄-alkylaminosulphonyl or C₁-C₄-alkylsulphonyl, where alkyl, alkylamino and alkylaminosulphonyl may be substituted by a substituent, the substituent being selected from the group consisting of hydroxy, amino, C₁-C₄-alkoxy, C₁-C₄-alkylamino, hydroxycarbonyl, aminocarbonyl, C₁-C₄-alkoxycarbonyl, C₁-C₄-alkylaminocarbonyl and a 5- or 6-membered heterocyclyl attached via a nitrogen atom, and where alkylaminocarbonyl may be substituted by a substituent, the substituent being selected from the group consisting of hydroxy, amino, C₁-C₄-alkylamino and a 5- or 6-membered heterocyclyl attached via a nitrogen atom,
R¹ represents hydrogen, cyano, hydroxy, C₁-C₄-alkyl, C₁-C₄-alkylcarbonyl, C₂-C7-cycloalkylcarbonyl, phenylcarbonyl, 4- to 7-membered heterocyclylcarbonyl or 5- or 6-membered heteroarylcarbonyl,
R² represents hydrogen, fluorine, chlorine, cyano, hydroxy, amino, trifluoromethyl, trifluoromethoxy, C₁-C₄-alkyl, C₁-C₄-alkoxy, C₁-C₄-alkoxymethyl, C₁-C₄-alkylamino, C₃-C₆-cycloalkyl, aminocarbonyl, C₁-C₄-alkoxycarbonyl or C₁-C₄-alkylaminocarbonyl,
R³ represents hydrogen, fluorine, chlorine, cyano, hydroxy, amino, trifluoromethyl, trifluoromethoxy, C₁-C₄-alkyl, C₁-C₄-alkoxy, C₁-C₄-alkoxymethyl, C₁-C₄-alkylamino, C₃-C₆-cycloalkyl, aminocarbonyl, C₁-C₄-alkoxycarbonyl or C₁-C₄-alkylaminocarbonyl,
R⁴ represents a group of the formula 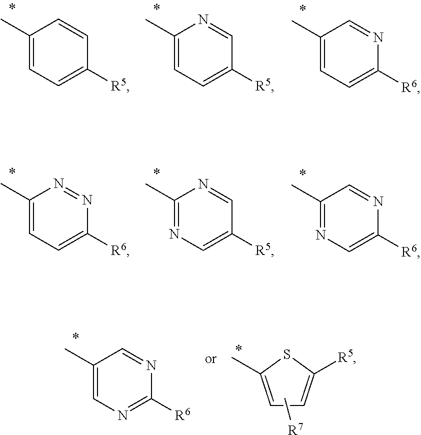
where * is the point of attachment to the carbonyl group, R⁵ represents hydrogen, fluorine, chlorine, cyano, ethynyl, C₁-C₄-alkyl, C₁-C₄-alkoxy or C₃-C₆-cycloalkyl, R⁶ represents hydrogen, amino, C₁-C₄-alkyl, C₁-C₄-alkylamino or C₃-C₆-cycloalkyl, and R⁷ represents hydrogen, fluorine, chlorine, amino or C₁-C₄-alkyl, or a salt thereof.

2. Compound according to claim 1, characterized in that
n represents the number 1, 2 or 3
A represents a 5-membered heteroaryl or partially unsaturated 5-membered heterocyclyl, where heteroaryl and heterocyclyl are attached in the 1- or 2-position to the phenyl ring and heteroaryl and heterocyclyl for their part have a 1,3-attachment to the phenyl ring and the carbonylaminomethyl group, and where heteroaryl and heterocyclyl may be substituted by a substituent R⁸, where R⁸ is attached to the neighbouring atom of the atom to which the carbonylaminomethyl group is attached and has a 1,4-attachment to the phenyl ring and where the atom to which R⁸ is attached is a nitrogen or carbon atom and where R⁸ represents amino, C₁-C₄-alkyl, C₁-C₄-alkoxy, C₁-C₄-alkoxymethyl, C₁-C₄-alkylamino, C₁-C₄-alkylaminomethyl, hydroxycarbonyl, hydroxycarbonylmethyl, hydroxycarbonylethyl, aminocarbonyl, aminocarbonylmethyl, aminocarbonylethyl, C₁-C₄-alkoxycarbonyl, C₁-C₄-alkoxycarbonylmethyl, C₁-C₄-alkoxycarbonylethyl, C₁-C₄-alkylaminocarbonyl, C₁-C₄-alkylaminocarbonylmethyl, alkylaminocarbonylethyl, aminosulphonyl, C₁-C₄-alkylaminosulphonyl or C₁-C₄-alkylsulphonyl, where alkyl may be substituted by a substituent, the substituent being selected from the group consisting of hydroxy and amino and where ethylaminocarbonyl and propylaminocarbonyl may be substituted by a substituent, the substituent being selected from the group consisting of hydroxy, amino and C₁-C₄-alkylamino,
R¹ represents hydrogen, cyano, hydroxy or C₁-C₄-alkyl,
R² represents hydrogen, fluorine, chlorine, cyano, C₁-C₄-alkyl or C₁-C₄-alkoxy,
R³ represents hydrogen, fluorine, chlorine, cyano, hydroxy, C₁-C₄-alkyl, C₁-C₄-alkoxy, C₁-C₄-alkoxymethyl, cyclopropyl, aminocarbonyl, C₁-C₄-alkoxycarbonyl or C₁-C₄-alkylaminocarbonyl,
R⁴ represents a group of the formula 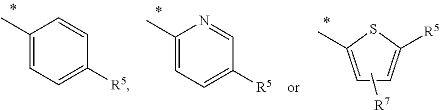
where * is the point of attachment to the carbonyl group, R⁵ represents fluorine, chlorine, ethynyl, methyl or methoxy, and R⁷ represents hydrogen.

3. Compound according to claim 1, characterized in that
n represents the number 1 or 2,
A represents a group of the formula 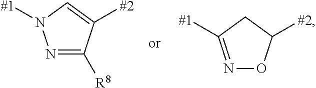
where #1 is the point of attachment to the phenyl ring and is attached in the 1-position to the phenyl ring, #2 is the point of attachment to the carbonylaminomethyl group, R⁸ represents hydrogen, C₁-C₄-alkyl, C₁-C₄-alkoxy, C₁-C₄-alkoxymethyl, C₁-C₄-alkylamino, C₁-C₄-alkylaminomethyl, hydroxycarbonyl, hydroxycarbonylmethyl, aminocarbonyl, aminocarbonylmethyl, C₁-C₄-alkoxycarbonyl, C₁-C₄-alkoxycarbonylmethyl, C₁-C₄-alkylaminocarbonyl or C₁-C₄-alkylaminocarbonylmethyl, where alkyl may be substituted by a substituent, where the substituent is selected from the group consisting of hydroxy and amino, and where ethylaminocarbonyl may be substituted by a substituent, where the substituent is selected from the group consisting of hydroxy, amino and C₁-C₄-alkylamino,
R¹ represents hydrogen,
R² represents hydrogen or fluorine,
R³ represents hydrogen, fluorine, chlorine, cyano, methyl, ethyl, n-propyl, isopropyl, methoxy, ethoxy, methoxymethyl or cyclopropyl,
R⁴ represents a group of the formula 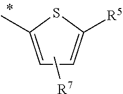
where * is the point of attachment to the carbonyl group, R⁵ represents fluorine, chlorine or methyl, and R⁷ represents hydrogen.

4. Compound according to claim 1, characterized in that
n represents the number 1,
A represents a group of the formula 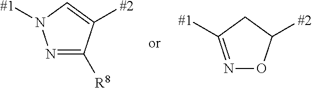
where # is the point of attachment to the phenyl ring and is attached in the 1-position to the phenyl ring, #2 is the point of attachment to the carbonylaminomethyl group, R⁸ represents hydrogen, hydroxymethyl, aminomethyl, C₁-C₄-alkyl, C₁-C₄-alkoxy, C₁-C₄-alkoxymethyl, C₁-C₄-alkylaminomethyl, hydroxycarbonyl, aminocarbonyl, C₁-C₄-alkoxycarbonyl, C₁-C₄-alkylaminocarbonyl, hydroxyethylaminocarbonyl or C₁-C₄-alkylaminoethylamino carbonyl,
R¹ represents hydrogen,
R² represents hydrogen or fluorine,
R³ represents hydrogen, fluorine, chlorine, methyl or methoxy,
R⁴ represents a group of the formula 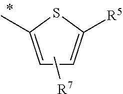
where * is the point of attachment to the carbonyl group, R⁵ represents chlorine, and R⁷ represents hydrogen.

5. Process for preparing a compound of the formula (I) or one of its salts, its solvates or the solvates of its salts according to claim 1, characterized in that [A] a compound of the formula 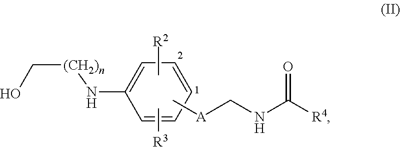
in which n, A, R², R³ and R⁴ have the meaning given in claim 1 is reacted in an inert solvent in the presence of an acid with cyanogen bromide to give a compound of the formula (I) in which
R¹ represents hydrogen, or
[B] the compound of the formula 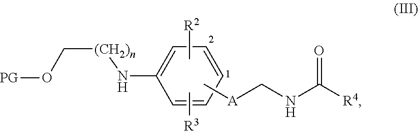
in which n, A, R², R³ and R⁴ have the meaning given in claim 1 and PG represents a hydroxy protective group, preferably trimethylsilyl or tertbutyldimethylsilyl, is reacted in a′ three-step process initially in an inert solvent with cyanogen bromide, preferably in the presence of a base, to give a compound of the formula 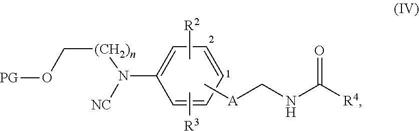
in which n, A, R², R³ and R⁴ have the meaning given in claim 1 and PG represents a hydroxy protective group, preferably trimethylsilyl or tertbutyldimethylsilyl, and then converted by removal of the protective group PG into a compound of the formula 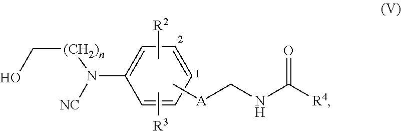
in which n, A, R², R³ and R⁴ have the meaning given in claim 1, and in the third step the compound of the formula (V) is cyclized in an inert solvent in the presence of an acid to give a compound of the formula (I), in which R¹ represents hydrogen, the removal of the protective group and the cyclization preferably being carried out in one reaction step, or
[C] the compound of the formula (II) is reacted in the first step with a compound of the formula 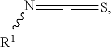
in which R¹ represents C₁-C₁-alkyl, C₁-C₄-alkylcarbonyl, C₃-C₇-cycloalkylcarbonyl, phenylcarbonyl, 4- to 7-membered heterocyclylcarbonyl or 5- or 6-membered heteroaryl carbonyl, and cyclized in the second step, or
[D] the compound of the formula (II) is reacted with a compound of the formula 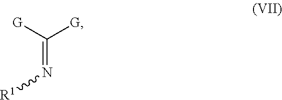
in which R¹ represents cyano or C₁-C₄-alkyl and G represents a leaving group, preferably phenoxy or methylthio, or
[E] the compound of the formula (I) in which R¹ represents hydrogen is reacted with hydroxylamine hydrochloride to give a compound of the formula (I) in which R¹ represents hydroxy.

6-9. (canceled)

10. Medicament, comprising a compound according to claim 1 in combination with an inert non-toxic pharmaceutically acceptable auxiliary.

11-12. (canceled)

13. Method for the treatment and/or prophylaxis of thromboembolic disorders in humans and animals using an anticoagulatory effective amount of at least one compound according to claim 1.

14. Method for preventing blood coagulation in vitro, characterized in that an anticoagulatory effective amount of a compound according to claim 1.
